(19) United States
(12) Patent Application Publication
(10) Pub. No.: US 2007/0081964 A1
(43) Pub. Date: Apr. 12, 2007

(54) Use of polymers based on n-vinyl caprolactam in hair cosmetics

(75) Inventors: Gabi Müller, Mannheim (DE); Peter Hossel, Schifferstadt (DE); Claudia Wood, Weinheim (DE)

Correspondence Address: CONNOLLY BOVE LODGE & HUTZ, LLP, P O BOX 2207, WILMINGTON, DE, 19899, US

(73) Assignee: BASF Aktiengesellschaft Patents, Trademarks and Licenses, Ludwigshafen (DE)

(21) Appl. No.: 10/582,110

(22) PCT Filed: Dec. 8, 2004
(86) PCT No.: PCT/EP04/13977

§ 371 Date: Jun. 8, 2006

(30) Foreign Application Priority Data
Dec. 8, 2003 (DE) 103 57 532.4

(51) Int. Cl. A61K 8/81

(52) U.S. Cl. 424/70.15

(57) ABSTRACT

Use of polymers of 30-65% by weight of N-vinylcaprolactam, 35-65% by weight of a monomer mixture of N-vinylimidazole and vinylpyrrolidone, where the weight ratio of N-vinylimidazole to vinylpyrrolidone is between 0.085 and 0.30, 0-15% by weight of a nitrogen-containing quaternizable monomer (A), which is different from N-vinylimidazole, where the sum of monomer (A) and N-vinylimidazole is less than 18% by weight, in cosmetic preparations.

The present invention relates to cosmetic preparations, in particular for hair cosmetic applications, which comprise copolymers based on N-vinyllactams and N-vinylheterocyclic compounds.

### PRIOR ART

Copolymers comprising N-vinyllactam, such as Luviskol K, Luviskol VA, Luviquat Hold or Luviskol Plus (BASF), are used in cosmetic, in particular hair cosmetic, preparations, in particular as hair-setting compositions.

For hair cosmetics, gel preparations are used to an increasing degree. Hair-setting agents in such gel preparations, and the gel preparation should satisfy the following requirements. Nontoxic, clear, colorless, non-sticky, good setting action, not very hygroscopic, good consistency.

Some of the copolymers used here still have properties which are in need of improvement. With the exception of a few nonionic copolymers, such as Luviskol K 90, K30, Luviskol VA 64 (BASF) or polyvinylformamide, most gels are cloudy to opaque. Likewise, the willingness to absorb water, and also the stickiness of the hair treated with these copolymers is too high. The setting effect is likewise still in need of improvement.

The polymers according to the invention do not have the above disadvantages. DE-C 12 61 822 describes copolymers of N-vinylcaprolactam with, for example, N-vinylimidazole and N-vinylpyrrolidone. The copolymers serve as agents for reducing pigment migration during the coloring of fiber material with pigment dye liquors.

WO 9831328 describes aqueous preparations comprising (a) 0.1 to 10% by weight of a copolymer based on N-vinylcaprolactam, N-vinylimidazole, N-vinylpyrrolidone and (b) 0.1 to 10% by weight of at least one polyoxyethylene C₆-C₁₅-monoalkyl ether, and their use in cosmetic formulations.

EP 0709411 describes soluble copolymers with 15 to 84.99% by weight of at least one monomer from the group of N-vinylcaprolactam, N-vinylimidazole, N-vinylpyrrolidone in alcoholic solution.

EP 0455081A1 describes, inter alia, copolymers of
35 to 65% by weight of N-vinylcaprolactam,
35 to 65% by weight of a mixture of 5 to 50 parts by weight of N-vinylimidazole and 10 to 60 parts by weight of N-vinylpyrrolidone (which corresponds to a VI:VP (VI/VP) ratio between 1:12 (1/12) and 5:1 (5/1))
0 to 4% by weight of further free-radically copolymerizable monomers,
   and also the use of such copolymers as hair-setting and haircare compositions.

Table 3 discloses polymers of 35% by weight of vinylcaprolactam, 50% by weight of VI and 15% by weight of VP (example 8) or of 60% by weight of vinylcaprolactam, 30% by weight of VI and 10% by weight of VC (example 9). Here, the VI/VP ratio is 3.3:1 or 3:1, respectively.

It was the object to provide polymers for use in cosmetics, in particular hair cosmetics, which are characterized by a high setting action and can be used particularly advantageously in hair gel preparations since they are clear, colorless and non-sticky.

We have now found the use of polymers of
30 to 65% by weight of N-vinylcaprolactam,
35 to 65% by weight of a monomer mixture of N-vinylimidazole and N-vinylpyrrolidone, where the weight ratio of N-vinylimidazole to N-vinylpyrrolidone is between 0.085 and 0.30,
0 to 15% by weight of a nitrogen-containing quaternizable monomer (A), which is different from N-vinylimidazole, where the sum of monomer (A) and N-vinylimidazole is less than 18% by weight,
   in cosmetic preparations.

Preference is given to using polymers of
35 to 45% by weight of N-vinylcaprolactam,
55 to 65% by weight of a monomer mixture of N-vinylimidazole and vinylpyrrolidone, where the weight ratio of N-vinylimidazole to vinylpyrrolidone is between 0.1 and 0.2, particularly preferably 0.15 and 0.18,
   in hair cosmetic preparations, in particular hair gels.

The monomers (A) which may be used are the following monomers:

N,N-dialkylaminoalkyl acrylates and methacrylates and N-dialkylaminoalkylacrylamides and -methacrylamides of the general formula (VII) 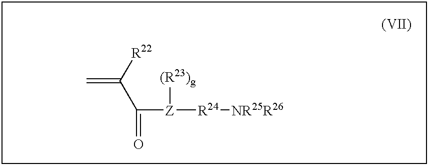
where
R²²=H, alkyl having 1 to 8 carbon atoms,
R²³=H, methyl,
R²⁴=alkylene having 1 to 24 carbon atoms, optionally substituted by alkyl,
R²⁵, R²⁶=C₁-C₄₀ alkyl radical,
Z=nitrogen when g=1 or oxygen when g=0

The amides may be present in unsubstituted form, N-alkyl- or N-alkylamino-monosubstituted form or N,N-dialkyl-substituted or N,N-dialkylamino-disubstituted form, in which the alkyl or alkylamino groups are derived from C₁-C₄₀ linear, C₃-C₄₀ branched-chain, or C₃-C₄₀ carbocyclic units. Additionally, the alkylamino groups may be quaternized.

Preferred comonomers of the formula VII are N,N-dimethylaminomethyl(meth)acrylate, N,N-diethylaminomethyl(meth)acrylate, N,N-dimethylaminoethyl(meth)acrylate, N,N-diethylaminoethyl(meth)acrylate, N-[3-(dimethylamino)propyl]methacrylamide and N-[3-(dimethylamino)propyl]acrylamide.

Also suitable are N-vinylimidazole derivatives of the general formula VIII as monomer (A), in which R²⁷ to R²⁹, independently of one another, are hydrogen, C₁-C₄-alkyl or phenyl, with the proviso that R²⁷, R²⁸ and R²⁹ must not be hydrogen at the same time:

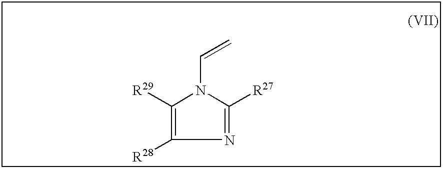

Further suitable monomers (A) are diallylamines of the general formula (IX) 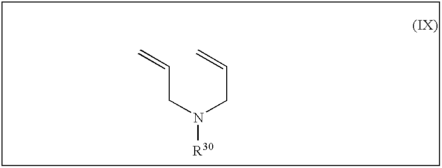
where R³⁰=C₁- to C₂₄-alkyl

The monomers (A) may also be used in quaternized form, or be quaternized after polymerization has taken place.

Suitable for the quaternization of the amines are, for example, alkyl halides having 1 to 24 carbon atoms in the alkyl group, e.g. methyl chloride, methyl bromide, methyl iodide, ethyl chloride, ethyl bromide, propyl chloride, hexyl chloride, dodecyl chloride, lauryl chloride and benzyl halides, in particular benzyl chloride and benzyl bromide. Further suitable quaternizing agents are dialkyl sulfates, in particular dimethyl sulfate or diethyl sulfate. The quaternization of the basic amines can also be carried out with alkylene oxides, such as ethylene oxide or propylene oxide, in the presence of acids. Preferred quaternizing agents are: methyl chloride, dimethyl sulfate or diethyl sulfate.

The quaternization can be carried out before the polymerization or after the polymerization.

The polymers are prepared by free-radical polymerization of the monomers. The procedure is carried out under the customary polymerization conditions, for example in accordance with the methods of precipitation, suspension, emulsion, solution or dispersion polymerization, and also bulk polymerization. Solution polymerization in water or an organic solvent, generally an alcohol, or in a water/alcohol mixture has proven to be particularly advantageous. These procedures are usually carried out at temperatures of from 60 to 130° C., it being possible to carry out the reaction at atmospheric pressure, autogenous pressure or reduced pressure.

Initiators for the free-radical polymerization which may be used are the water-soluble and water-insoluble peroxo and/or azo compounds customary for this purpose, for example alkali metal or ammonium peroxydisulfates, dibenzoyl peroxide, tert-butyl perpivalate, tert-butyl per-2-ethylhexanoate, di-tert-butyl peroxide, tert-butyl hydroperoxide, azobisisobutyronitrile, azobis(2-amidinopropane) dihydrochloride or 2,2′-azobis(2-methylbutyronitrile). Also suitable are initiator mixtures or redox initiator systems, such as, for example, ascorbic acid/iron (II) sulfate/sodium peroxodisulfate, tert-butyl hydroperoxide/sodium disulfite, tert-butyl hydroperoxide/sodium hydroxy-methanesulfinate. The initiators may be used in the customary amounts, for example 0.05 to 5% by weight, based on the amount of monomers to be polymerized.

The molecular weight and the K value of the polymers can be varied within a wide range in a manner known per se through the choice of polymerization conditions, for example polymerization time, polymerization temperature or initiator concentration, and through the content of crosslinker and regulator.

The K values of the polymers are in a range between 10 and 350, preferably 20 to 200 and particularly preferably 35 to 110, very particularly between 40 and 80. The K values are measured at 25° C. at 1% strength in aqueous solution in accordance with Fikentscher, Cellulosechemie, vol. 13, pp. 58-64 (1932).

The amount of monomers and solvents is advantageously chosen so that 20 to 40% strength by weight solutions of the copolymers are obtained.

The polymer mixture can optionally be subjected to an additional after-polymerization, and if appropriate to an after-treatment by steam distillation, treatment with acids/alkalis or oxidizing agents or reducing agents. In a preferred embodiment, the polymer is subjected to steam distillation.

For the stabilization, the polymer solution is treated with Euxyl® K 100 (Schülke & Mayr), with Phenonip® (Clariant), with ethanol or with an alternative stabilizer.

Pulverulent products can be obtained by precipitation, spray-drying from suitable solvent systems or freeze-drying.

The gels formulated with the described film formers are characterized by improved properties compared with the prior art.

The polymers according to the invention can advantageously be used in cosmetic preparations, in particular hair cosmetic preparations.

The term “cosmetic preparations” is to be understood in the wide sense and means all those preparations which are suitable for application to skin and/or hair and/or nails and have a purpose other than a merely medicinal-therapeutic one.

The polymers according to the invention can be used in skin cosmetic preparations.

For example, the polymers according to the invention are used in cosmetic compositions for cleansing the skin. Such cosmetic cleansing compositions are chosen from bar soaps, such as toilet soaps, curd soaps, transparent soaps, luxury soaps, deodorant soaps, cream soaps, baby soaps, skin protection soaps, abrasive soaps and syndets, liquid soaps, such as pasty soaps, soft soaps and washing pastes, and liquid washing, shower and bath preparations, such as washing lotions, shower baths and gels, foam baths, oil baths and scrub preparations.

Preferably, the polymers according to the invention are used in cosmetic compositions for the care and protection of the skin, in nailcare compositions and in preparations for decorative cosmetics.

Particular preference is given to the use in skincare compositions, personal hygiene compositions, footcare compositions, deodorants, photoprotective compositions, repellents, shaving compositions, hair-removal compositions, antiacne compositions, make-up, mascara, lipsticks, eyeshadows, kohl pencils, eyeliners, blushers, powders and eyebrow pencils.

The skincare compositions are in particular in the form of W/O or O/W skin creams, day and night creams, eye creams, face creams, antiwrinkle creams, moisturizing creams, bleaching creams, vitamin creams, skin lotions, care lotions and moisturizing lotions.

In the cosmetic preparations, the polymers according to the invention can develop their particular effects. The polymers can inter alia contribute to the moisturization and conditioning of the skin and to an improvement in the feel of the skin. The polymers can also act as thickeners in the formulations. By adding the polymers according to the invention, a considerable improvement in the skin compatibility can be achieved in certain formulations.

The copolymers according to the invention are present in the skin cosmetic preparations in an amount of from about 0.001 to 20% by weight, preferably 0.01 to 10% by weight, very particularly preferably 0.1 to 5% by weight, based on the total weight of the compositions.

Depending on the field of use, the compositions according to the invention can be applied in a form suitable for skincare, such as, for example, in the form of a cream, foam, gel, stick, powder, mousse, milk or lotion.

Besides the polymers according to the invention and suitable solvents, the skin cosmetic preparations can also comprise additives customary in cosmetics, such as emulsifiers, preservatives, perfume oils, cosmetic active ingredients, such as phytantriol, vitamin A, E and C, retinol, bisabolol, panthenol, photoprotective agents, bleaching agents, colorants, tints, tanning agents (e.g. dihydroxyacetone), collagen, protein hydrolysates, stabilizers, pH regulators, dyes, salts, thickeners, gel formers, bodying agents, silicones, humectants, refatting agents and further customary additives.

Suitable solvents are, in particular, water and lower monoalcohols or polyols having 1 to 6 carbon atoms or mixtures thereof; preferred monoalcohols or polyols are ethanol, isopropanol, propylene glycol, glycerol and sorbitol.

Further customary additives which may be present are fatty substances, such as mineral and synthetic oils, such as, for example, paraffins, silicone oils and aliphatic hydrocarbons having more than 8 carbon atoms, animal and vegetable oils, such as, for example, sunflower oil, coconut oil, avocado oil, olive oil, lanolin, or waxes, fatty acids, fatty acid esters, such as, for example, triglycerides of C₆-C₃₀-fatty acids, wax esters, such as, for example, jojoba oil, fatty alcohols, vaseline, hydrogenated lanolin and acetylated lanolin. Mixtures thereof can of course also be used.

Customary thickeners in such formulations are crosslinked polyacrylic acids and derivatives thereof, polysaccharides, such as xanthan gum, agar agar, alginates or tyloses, carboxymethylcellulose or hydroxycarboxymethylcellulose, fatty alcohols, monoglycerides and fatty acids, polyvinyl alcohol and polyvinylpyrrolidone.

The polymers according to the invention can also be mixed with conventional polymers if specific properties are to be established.

Suitable conventional polymers are, for example, anionic, cationic, amphoteric and neutral polymers.

Examples of anionic polymers are homopolymers and copolymers of acrylic acid and methacrylic acid or salts thereof, copolymers of acrylic acid and acrylamide and salts thereof; sodium salts of polyhydroxycarboxylic acids, water-soluble or water-dispersible polyesters, polyurethanes and polyureas. Particularly suitable polymers are copolymers of t-butyl acrylate, ethyl acrylate, methacrylic acid (e.g. Luvimer® 100P), copolymers of ethyl acrylate and met hacrylic acid (e.g. Luvimer® MAE), copolymers of N-tert-butyl-acrylamide, ethyl acrylate, acrylic acid (Ultrahold® 8, strong), copolymers of vinyl acetate, crotonic acid and if appropriate further vinyl esters (e.g. Luviset® grades), maleic anhydride copolymers, if appropriate reacted with alcohols, anionic polysiloxanes, e.g. carboxy functional ones, copolymers of vinylpyrrolidone, t-butyl acrylate, methacrylic acid (e.g Luviskol® VBM), copolymers of acrylic acid and methacrylic acid with hydrophobic monomers, such as, for example, C₄-C₃₀-alkyl esters of meth(acrylic acid), C₄-C₃₀-alkylvinyl esters, C₄-C₃₀-alkyl vinyl ethers and hyaluronic acid.

Further suitable polymers are cationic polymers with the INCI name polyquaternium, e.g. copolymers of vinylpyrrolidone/N-vinylimidazolium salts (Luviquat® FC, Luviquat HM, Luviquat® MS, Luviquat® Care), copolymers of N-vinylpyrrolidone/dimethylaminoethyl methacrylate, quaternized with diethyl sulfate (Luviquat® PQ 11), copolymers of N-vinylcaprolactam/N-vinylpyrrolidone/N-vinylimidazolium salts (Luviquat® Hold); cationic cellulose derivatives (polyquaternium-4 and -10), acrylamide copolymers (polyquaternium-7) and chitosan.

Suitable further polymers are also neutral polymers, such as polyvinylpyrrolidones, copolymers of N-vinylpyrrolidone and vinyl acetate and/or vinyl propionate, polysiloxanes, polyvinylcaprolactam and copolymers containing N-vinylpyrrolidone, polyethyleneimines and salts thereof, polyvinylamines and salts thereof, cellulose derivatives, polyaspartic acid salts and derivatives.

To establish certain properties, the preparations can additionally also comprise conditioning substances based on silicone compounds. Suitable silicone compounds are, for example, polyalkylsiloxanes, polyarylsiloxanes, polyarylalkylsiloxanes, polyether siloxanes or silicone resins.

The copolymers according to the invention are used in cosmetic preparations, the preparation of which takes place in accordance with the customary rules familiar to the person skilled in the art.

Such formulations are advantageously present in the form of emulsions, preferably in the form of water-in-oil (W/O) or oil-in-water (O/W) emulsions. It is, however, also possible and if appropriate advantageous according to the invention to choose other formulation types, for example hydrodispersions, gels, oils, oleogels, multiple emulsions, for example in the form of W/O/W or O/W/O emulsions, anhydrous ointments or ointment bases etc.

The preparation of emulsions which can be used according to the invention takes place by known methods.

Besides the copolymer according to the invention, the emulsions comprise customary constituents, such as fatty alcohols, fatty acid esters and, in particular, fatty acid triglycerides, fatty acids, lanolin and derivatives thereof, natural or synthetic oils or waxes and emulsifiers in the presence of water.

The selection of additives specific to the type of emulsion and the preparation of suitable emulsions is described, for example, in Schrader, Grundlagen und Rezepturen der Kosmetika [Fundamentals and Formulations of Cosmetics], Hüthig Buch Verlag, Heidelberg, 2nd edition, 1989, third part, to which express reference is hereby made.

Thus, a skin cream which can be used according to the invention can be in the form, for example, of a W/O emulsion. An emulsion of this type comprises an aqueous phase which is emulsified in an oil or fatty phase by means of a suitable emulsifier system.

The concentration of the emulsifier system in this type of emulsion is about 4 to 35% by weight, based on the total weight of the emulsion; the fatty phase constitutes about 20 to 60% by weight and the aqueous phase about 20 to 70% by weight, in each case based on the total weight of the emulsion. The emulsifiers are those which are customarily used in this type of emulsion. They are chosen, for example, from: C₁₂-C₁₈-sorbitan fatty acid esters; esters of hydroxystearic acid and C₁₂-C₃₀-fatty alcohols; mono- and diesters of C₁₂-C₁₈-fatty acids and glycerol or polyglycerol; condensates of ethylene oxide and propylene glycols; oxypropylenated/oxyethylenated C₁₂-C₂₀-fatty alcohols; polycyclic alcohols, such as sterols; aliphatic alcohols with a high molecular weight, such as lanolin; mixtures of oxypropylenated/polyglycerolated alcohols and magnesium isostearate; succinic esters of polyoxyethylenated or polyoxypropylenated fatty alcohols; and mixtures of magnesium, calcium, lithium, zinc or aluminum lanolate and hydrogenated lanolin or lanolin alcohol.

Suitable fatty components which may be present in the fatty phase of the emulsions include hydrocarbon oils, such as paraffin oil, purcellin oil, perhydrosqualene and solutions of microcrystalline waxes in these oils; animal or vegetable oils, such as sweet almond oil, avocado oil, calophylum oil, lanolin and derivatives thereof, castor oil, sesame oil, olive oil, jojoba oil, karité oil, hoplostethus oil; mineral oils whose distillation start point under atmospheric pressure is at about 250° C. and whose distillation end point is at 410° C., such as, for example, vaseline oil; esters of saturated or unsaturated fatty acids, such as alkyl myristates, e.g. isopropyl, butyl or cetyl myristate, hexadecyl stearate; ethyl or isopropyl palmitate, octanoic or decanoic acid triglycerides and cetyl ricinoleate.

The fatty phase may also comprise silicone oils soluble in other oils, such as dimethylpolysiloxane, methylphenylpolysiloxane and the silicone glycol copolymer, fatty acids and fatty alcohols.

In order to favor the retention of oils, it is also possible to use waxes, such as, for example, carnauba wax, candellila wax, beeswax, microcrystalline wax, ozokerite wax and Ca, Mg and Al oleates, myristates, linoleates and stearates.

In general, these water-in-oil emulsions are prepared by adding the fatty phase and the emulsifier to the batch container. The latter is heated at a temperature of from 70 to 75° C., then the oil-soluble ingredients are added, and water which has been heated beforehand to the same temperature and in which the water-soluble ingredients have been dissolved beforehand is added; the mixture is stirred until an emulsion of the desired fineness is achieved, which is then left to cool to room temperature, if appropriate with a lesser amount of stirring.

In addition, a care emulsion according to the invention may be in the form of an ONV emulsion. Such an emulsion usually comprises an oil phase, emulsifiers which stabilize the oil phase in the water phase, and an aqueous phase which is usually present in thickened form.

The aqueous phase of the O/W emulsion of the preparations according to the invention if appropriate comprises
alcohols, diols or polyols and ethers thereof, preferably ethanol, isopropanol, propylene glycol, glycerol, ethylene glycol monoethyl ether;
customary thickeners or gel formers, such as, for example, crosslinked polyacrylic acids and derivatives thereof, polysaccharides, such as xanthan gum or alginates, carboxymethylcellulose or hydroxycarboxymethylcellulose, fatty alcohols, polyvinyl alcohol and polyvinylpyrrolidone.

The oil phase comprises oil components customary in cosmetics, such as, for example:
esters of saturated and/or unsaturated, branched and/or unbranched C₃-C₃₀alkanecarboxylic acids and saturated and/or unsaturated, branched and/or unbranched C₃-C₃₀-alcohols, of aromatic carboxylic acids and saturated and/or unsaturated, branched and/or unbranched C₃-C₃₀-alcohols, for example isopropyl myristate, isopropyl stearate, hexyldecyl stearate, oleyl oleate; and also synthetic, semisynthetic and natural mixtures of such esters, such as jojoba oil;
branched and/or unbranched hydrocarbons and hydrocarbon waxes;
silicone oils, such as cyclomethicone, dimethylpolysiloxane, diethylpolysiloxane, octamethylcyclotetrasiloxane and mixtures thereof;
dialkyl ethers;
mineral oils and mineral waxes;
triglycerides of saturated and/or unsaturated, branched and/or unbranched C₈-C₂₄-alkanecarboxylic acids; they can be chosen from synthetic, semisynthetic or natural oils, such as olive oil, palm oil, almond oil or mixtures.

Suitable emulsifiers are, preferably, O/W emulsifiers, such as polyglycerol esters, sorbitan esters or partially esterified glycerides.

The preparation may be carried out by melting the oil phase at about 80° C.; the water-soluble constituents are dissolved in hot water, added to the oil phase slowly and with stirring; homogenized and stirred until cold.

The polymers according to the invention are also suitable for use in washing and shower gel formulations and bath preparations.

As well as the polymers according to the invention, such formulations usually comprise anionic surfactants as base surfactants and amphoteric and nonionic surfactants as cosurfactants, and also lipids, perfume oils, dyes, organic acids, preservatives and antioxidants, and thickeners/gel formers, skin conditioning agents and humectants.

In the washing, showering and bath preparations, all anionic, neutral, amphoteric or cationic surfactants customarily used in body-cleansing compositions may be used.

The formulations comprise 2 to 50% by weight of surfactants, preferably 5 to 40% by weight, particularly preferably 8 to 30% by weight.

Suitable anionic surfactants are, for example, alkyl sulfates, alkyl ether sulfates, alkylsulfonates, alkylarylsulfonates, alkyl succinates, alkyl sulfosuccinates, N-alkoylsarcosinates, acyl taurates, acyl isethionates, alkyl phosphates, alkyl ether phosphates, alkyl ether carboxylates, alpha-olefinsulfonates, in particular the alkali metal and alkaline earth metal salts, e.g. sodium, potassium, magnesium, calcium, and ammonium and triethanolamine salts. The alkyl ether sulfates, alkyl ether phosphates and alkyl ether carboxylates can have between 1 and 10 ethylene oxide or propylene oxide units, preferably 1 to 3 ethylene oxide units, in the molecule. Suitable compounds are, for example, sodium lauryl sulfate, ammonium lauryl sulfate, sodium lauryl ether sulfate, ammonium lauryl ether sulfate, sodium lauryl sarcosinate, sodium oleyl succinate, ammonium lauryl sulfosuccinate, sodium dodecylbenzenesulfonate, triethanolamine dodecylbenzenesulfonate.

Suitable amphoteric surfactants are, for example, alkylbetaines, alkylamidopropyl-betaines, alkylsulfobetaines, alkyl glycinates, alkyl carboxyglycinates, alkyl amphoacetates or amphopropionates, alkyl amphodiacetates or amphodipropionates.

For example, it is possible to use cocodimethylsulfopropylbetaine, laurylbetaine, cocamidopropylbetaine or sodium cocoamphopropionate.

Suitable nonionic surfactants are, for example, the reaction products of aliphatic alcohols or alkylphenols, having 6 to 20 carbon atoms in the alkyl chain, which may be linear or branched, with ethylene oxide and/or propylene oxide. The amount of alkylene oxide is about 6 to 60 moles per mole of alcohol. Also suitable are alkylamine oxides, mono- or dialkylalkanolamides, fatty acid esters of polyethylene glycols, ethoxylated fatty acid amides, alkyl polyglycosides or sorbitan ether esters.

In addition, the washing, shower and bath preparations can comprise customary cationic surfactants, such as, for example, quaternary ammonium compounds, for example cetyltrimethylammonium chloride.

Additionally, further customary cationic polymers may also be used, such as, for example, copolymers of acrylamide and dimethyldiallylammonium chloride (polyquaternium-7), cationic cellulose derivatives (polyquaternium-4, -10), guar hydroxypropyltrimethylammonium chloride (INCI: Hydroxypropyl Guar Hydroxypropyltrimonium Chloride), copolymers of N-vinylpyrrolidone and quaternized N-vinylimidazole (polyquaternium-16, -44, -46), copolymers of N-vinylpyrrolidone/dimethylaminoethyl methacrylate, quaternized with diethyl sulfate (polyquaternium-11) and others.

In addition, the washing and shower gel formulations and bath preparations can comprise thickeners, such as, for example, sodium chloride, PEG-55, propylene glycol oleate, PEG-120 methyl glucose dioleate and others, and also preservatives, further active ingredients and auxiliaries and water.

Hair cosmetic preparations comprise, in particular, styling compositions and/or conditioning agents in hair cosmetic preparations, such as hair treatments, hair mousses, (hair) gels or hair sprays, hair lotions, hair rinses, hair shampoos, hair emulsions, hair-end fluids, neutralizers for permanent waves, hair colorants and bleaches, hot-oil treatment preparations, conditioners, setting lotions or hair sprays. Depending on the field of application, the hair cosmetic preparations can be applied in the form of (aerosol) spray, (aerosol) mousse, gel, gel spray, cream, lotion or wax.

In a preferred embodiment, the hair cosmetic formulations according to the invention comprise

a) 0.05 to 20% by weight of the polymer according to the invention

b) 20 to 99.95% by weight of water and/or alcohol

c) 0 to 79.5% by weight of further constituents.

Alcohol is understood as meaning all alcohols customary in cosmetics, e.g. ethanol, isopropanol, n-propanol.

Further constituents are understood as meaning the additives customary in cosmetics, for example propellants, antifoams, interface-active compounds, i.e. surfactants, emulsifiers, foam formers and solubilizers. The interface-active compounds used may be anionic, cationic, amphoteric or neutral. Further customary constituents may also be, for example, preservatives, perfume oils, opacifiers, active ingredients, UV filters, care substances, such as panthenol, collagen, vitamins, protein hydrolysates, alpha- and beta-hydroxycarboxylic acids, protein hydrolysates, stabilizers, pH regulators, dyes, viscosity regulators, gel formers, dyes, salts, humectants, refatting agents, complexing agents and further customary additives.

Gelling agents which may be used are all gelling agents customary in cosmetics. These include slightly crosslinked polyacrylic acid, for example Carbomer (INCI) or Acrylates/C10-30 Alkyl Acrylate Crosspolymer (INCI), Acrylates/Beheneth-25 Methacrylate Copolymer (INCI), PVM/MA decadiene crosspolymer, cellulose derivatives, e.g. hydroxypropylcellulose, hydroxyethylcellulose, cationically modified celluloses, polysaccharides, e.g. xanthum gum, hydroxypropyl starch phosphate, potato starch modified, caprylic/capric triglyceride, sodium acrylates copolymer, Polyquaternium-32 (and) Paraffinum Liquidum (INCI), Sodium Acrylates Copolymer (and) Paraffinum Liquidum (and) PPG-1 Trideceth-6, acrylamidopropyltrimonium chloride/acrylamide copolymer, steareth-10 allyl ether acrylates copolymer, Polyquaternium-37 (and) Paraffinum Liquidum (and) PPG-1 Trideceth-6, polyacrylamides and C13-14 isoparaffin and laureth-7, C13-14 isoparaffin and mineral oil and sodium polyacrylate and polyacrylamide and polysorbate 85, C13-14 isoparaffin and isostearyl isostearate and sodium polyacrylate and polyacrylamide and polysorbate 60, acrylates/aminoacrylates/C10-30 alkyl PEG-20 itaconate copolymer, acrylates/steareth-20 itaconate copolymer, acrylates/ceteth-20 itaconate copolymer, polyquaternium 37 (and) propylene glycol dicaprate dicaprylate (and) PPG-1 trideceth-6, polyquaternium-7, polyquaternium-44.

Also included here are all styling and conditioning polymers known in cosmetics which can be used in combination with the polymers according to the invention if very specific properties are to be set.

Examples of suitable conventional hair cosmetic polymers are anionic polymers. Such anionic polymers are homopolymers and copolymers of acrylic acid and methacrylic acid or salts thereof, copolymers of acrylic acid and acrylamide and salts thereof; sodium salts of polyhydroxycarboxylic acids, water-soluble or water-dispersible polyesters, polyurethanes (Luviset® P.U.R.) and polyureas. Particularly suitable polymers are copolymers of t-butyl acrylate, ethyl acrylate, methacrylic acid (e.g. Luvimer® 100P), copolymers of N-tert-butylacrylamide, ethyl acrylate, acrylic acid (Ultrahold® 8, strong), copolymers of vinyl acetate, crotonic acid and if appropriate further vinyl esters (e.g. Luviset® grades), maleic anhydride copolymers, if appropriate reacted with alcohols, anionic polysiloxanes, e.g. carboxyfunctional ones, copolymers of vinylpyrrolidone, t-butyl acrylate, methacrylic acid (e.g. Luviskol® VBM).

In addition, the group of polymers suitable for the combination with the polymers according to the invention comprises, by way of example, Balancer CR (National Starch; acrylate copolymer), Balancer 0/55 (National Starch; acrylate copolymer), Balancer 47 (National Starch; octylacrylamide/acrylate/butylaminoethyl methacrylate copolymer), Aquaflex® FX 64 (ISP; isobutylene/ethylmaleimide/hydroxyethylmaleimide copolymer), Aquaflex® SF-40 (ISP/National Starch; VP/vinylcaprolactam/DMAPA acrylate copolymer), Allianz⁵⁸ LT-120 (ISP/Rohm & Haas; acrylate/C1-2 succinate/hydroxyacrylate copolymer), Aquarez® HS (Eastman; polyester-1), Diaformer® Z-400 (Clariant; methacryloylethylbetaine/methacrylate copolymer), Diaformere® Z-711 (Clariant; methacryloylethyl N-oxide/methacrylate copolymer), Diaformere® Z-712 (Clariant; methacryloylethyl N-oxide/methacrylate copolymer), Omnireze® 2000 (ISP; monoethyl ester of poly(methyl vinyl ether/maleic acid in ethanol), Amphomer® HC (National Starch; acrylate/octylacrylamide copolymer), Amphomer® 28-4910 (National Starch; octylacrylamide/acrylate/butylaminoethyl methacrylate copolymer), Advantage® HC 37 (ISP; terpolymer of vinylcaprolactam/vinylpyrrolidone/dimethylaminoethyl methacrylate), Acudyner 258 (Rohm & Haas; acrylate/hydroxy ester acrylate copolymer), Luviset® PUR (BASF, polyurethane-1), Luviflex® Silk (BASF), Eastman® AQ48 (Eastman).

Very particularly preferred anionic polymers are acrylates with an acid number greater than or equal to 120 and copolymers of t-butyl acrylate, ethyl acrylate, methacrylic acid.

Further suitable hair cosmetic polymers are cationic polymers with the INCI name Polyquaternium, e.g. copolymers of vinylpyrrolidone/N-vinylimidazolium salts (Luviquat® FC, Luviquat® HM, Luviquat® MS, Luviquat® Care), copolymers of N-vinyl-pyrrolidone/dimethylaminoethyl methacrylate, quaternized with diethyl sulfate (Luviquat® PQ 11), copolymers of N-vinylcaprolactam N-vinylpyrrolidone/N-vinyl-imidazolium salts (Luviquat® Hold); cationic cellulose derivatives (polyquaternium-4 and -10), acrylamide copolymers (polyquaternium-7).

In addition, cationic guar derivatives, such as guar hydroxypropyltrimonium chloride (INCI) may be used.

Further suitable hair cosmetic polymers are also neutral polymers, such as polyvinylpyrrolidones, copolymers of N-vinylpyrrolidones and vinyl acetate and/or vinyl propionate, polysiloxanes, polyvinylcaprolactam and copolymers containing N-vinylpyrrolidone, polyethyleneimines and salts thereof, polyvinylamines and salts thereof, cellulose derivatives, polyaspartic acid salts and derivatives.

To set certain properties, the preparations may additionally also comprise conditioning substances based on silicone compounds. Suitable silicone compounds are, for example, polyalkylsiloxanes, polyarylsiloxanes, polyarylalkylsiloxanes, polyether siloxanes, silicone resins or dimethicone copolyols (CTFA) and aminofunctional silicone compounds such as amodimethicones (CTFA).

The polymers according to the invention are particularly suitable as setting agents in hairstyling preparations, in particular hairsprays (aerosol sprays and pump sprays without propellant gas) and hair mousses (aerosol mousses and pump mousses without propellant gas).

### EXAMPLES

The polymers were prepared by the customary methods of free-radical polymerization in water. As a representative of all other preparation procedures, the synthesis of the polymers I may be described below.

### Example 1

Preparation of a polymer of 52% by weight of N-vinylpyrrolidone, 40% by weight of vinylcaprolactam and 8% by weight of vinylimidazole.

| | | |
|---|---|---|
| Initial charge: | 350 | g of demineralized water |
| Feed 1: | 156 | g of vinylpyrrolidone |
| | 24 | g of vinylimidazole |
| | 120 | g of vinylcaprolactam |
| | 400 | g of demineralized water |

The monomer mixture in feed 1 is adjusted to a pH of 7 using 10% strength phosphoric acid.

| | | |
|---|---|---|
| Feed 2: | 1 | g of Wako V 50 |
| | 50 | g of water |

The initial charge is heated to 75° C. While retaining the polymerization temperature, feed 1 is added over the course of 3 hours and, starting at the same time as feed 1, feed 2 is added over the course of 4 hours. The mixture is then after-polymerized for 2 h at an internal temperature of 75° C.

### Example 2

A polymer of 54% by weight of N-vinylpyrrolidone, 40% by weight of vinylcaprolactam and 6% by weight of vinylimidazole was prepared analogously to example 1.

### Example 3

A polymer of 59% by weight of N-vinylpyrrolidone, 35% by weight of vinylcaprolactam and 6% by weight of vinylimidazole was prepared analogously to example 1.

### Example 4

A polymer of 57% by weight of N-vinylpyrrolidone, 35% by weight of vinylcaprolactam and 8% by weight of vinylimidazole was prepared analogously to example 1.

### Example 5

A polymer of 49% by weight of N-vinylpyrrolidone, 45% by weight of vinylcaprolactam and 6% by weight of vinylimidazole was prepared analogously to example 1.

Examples 6-8 were prepared in accordance with the examples above.

Performance assessments were carried out using Ultrez 21 (Noveon) gels, which were formulated in accordance with the preparation procedures given.

The setting action was assessed by sensory assessment by trained individuals, and by measuring the flexural rigidity. The flexural rigidity was determined on tresses of hair treated with gel.

Curl Retention from the Gel

The curl retention was determined on tresses of hair treated with gel. The assessment was made at 25° C. and a defined atmospheric humidity of 90%.

TABLE 1

| | Composition of | | Setting, | Flexural | CR [%] gel |
|---|---|---|---|---|---|
| | the polymer | Appearance | sensory | rigidity | 25° C. |
| Sample | [% by wt.] | of the gel | assessment | [cN] | 90% r.h. |
| Luviskol K30 | Vinyl-pyrrolidone | Standard | Poor | 70-80 | 47 |
| (BASF) *) | (100%) | | | | |
| Luviskol K90 | Vinyl-pyrrolidone | Poorer than | Good | 125-145 | 61 |
| (BASF) *) | (100%) | standard | | | |
| B8 | VP/VI/VCAP | Better than | Good | 200 | 68 |
| | 57.5/2.5/40 | standard | | | |
| B7 | VP/VI/VCAP | Standard | Good | 232 | 88 |
| | 56/4/40 | | | | |
| B1 | VP/VI/VCAP | Standard | Very good | 270-326 | 70 |
| | 52/8/40 | | | | |
| B2 | VP/VI/VCAP | Standard | Very good | 250-290 | 80 |
| | 54/6/40 | | | | |
| B3 | VP/VI/VCAP | Standard | Very good | — | — |
| | 59/6/35 | | | | |
| B4 | VP/VI/VCAP | Standard | Very good | — | — |
| | 49/6/45 | | | | |
| B6 | VP/VI/VCAP | Significantly | — | — | — |
| | 40/20/40 | cloudy | | | |
| C1 **) | VP/VI/VCAP | Significantly | — | — | — |
| | 15/50/35 | cloudy | | | |
| C2 **) | VP/VI/VCAP | Significantly | — | — | — |
| | 10/30/60 | cloudy | | | |

*): Comparative example
**) Comparative examples from EP 0455081

Application

Preparation Procedures:

Carbopol gel (200 g)
3% polymer in 0.5% Carbopol (940 or Ultrez 21)

| | | |
|---|---|---|
| Batch a): | 98.68 g | of Carbopol stock solution (1% strength preserved |
| | | with Euxyl K 100) |
| | 1.32 g | of triethanolamine in 250 ml beaker |

The TEA is incorporated into the stock solution using a stirrer (about 90 rpm) until the mixture is clear (about 15 min)

| | | | |
|---|---|---|---|
| Batch b) | 6.00 | g | of polymer (solid) |
| | ad 100 | g | of water dist. in 250 ml Erlenmeyer |

If batch b) is completely dissolved, it is slowly incorporated into batch a) using a dropping funnel (about 1 drop/sec) at the same stirrer speed. If, after adding the solution dropwise, the finished gel is formed, it is after-stirred for about a further 30 minutes.

Shampoo

Preparation:

Dissolve all components in water, adjust pH and then add thickener.

Aerosol Spray

Preparation:

Weigh in all components. Adjust the pH, and bottle clear solution in pressurized container with propellant gas.

Wax

Preparation:

Weigh in the components of the phase, melt and stir evenly.

Aerosol Mousse

Preparation:

Mix perfume oil phase. Add the components of the aqueous-ethanolic phase one after the other and mix. If stated, add thickener and stir until evenly distributed. Adjust the pH. Bottle in a pressurized container with propellant gas.

Pump spray

Preparation:

Stir aqueous phase. Add the components of the ethanolic phase one after the other and distribute uniformly. Then bottle everything in a pump spray bottle.

Pump mousse

Preparation:

Prepare a uniform mixture from the components and bottle in a pump mousse bottle.

Emulsions of the O/W Type (Hair Rinses, Etc.)

Preparation:

Mix oil phase with emulsifiers (if necessary at elevated temperature) and add aqueous phase (with possible thickener, if necessary at elevated temperature) with stirring and homogenize.

Formulations:

Hair gel containing polymer as in Ex. B1 to B8 and Luviskol K30

| % | Raw material | Supplier | INCl |
|---|---|---|---|
| 0.50 | Carbopol 940 | (6) | Carbomer |
| 87.60 | Water, demin. | | Aqua dem. |
| 0.70 | Triethanolamine Care | (1) | Triethanolamine |
| 6.00 | Polymer B1 | (1) | |
| 5.00 | Luviskol K30 solution | (1) | PVP |
| q.s. | Perfume oil | | |
| q.s. | Cremophor RH 40 | (1) | PEG-40 Hydrogenated |
| | | | Castor Oil |
| 0.10 | Euxyl K100 | (42) | Benzyl Alcohol, Methyl- |
| | | | chloroisothiazolinone, |
| | | | Methylisothiazolinone |
| 0.10 | Vitamin E acetate | | Tocopheryl Acetate |

Suppliers
(1) BASF Aktiengesellschaft
(6) B. F. Goodrich Company Chemical Division
(42) Schülke & Mayr GmbH

Hair gel containing polymer as in Ex. B1 to B8 and Luviskol VA64

| % | Raw material | Supplier | INCl |
|---|---|---|---|
| 0.50 | Carbopol 980 | (6) | Carbomer |
| 87.60 | Water, demin. | | Aqua dem. |
| 0.90 | Neutrol TE | (1) | Tetrahydroxypropyl |
| | | | Ethylenediamine |
| 7.00 | Polymer B1 | (1) | |
| 4.00 | Luviskol VA64 W | (1) | VP/VA Copolymer |
| q.s. | Perfume oil | | |
| q.s. | Cremophor CO 40 | (1) | PEG-40 Hydrogenated |
| | | | Castor Oil |
| 0.10 | Euxyl K100 | (42) | Benzyl Alcohol, Methyl- |
| | | | chloroisothiazolinone, |
| | | | Methylisothiazolinone |
| 0.10 | 1,2-Propylene | (1) | Propylene Glycol |
| | glycol Care | | |

Suppliers
(1) BASF Aktiengesellschaft
(6) B. F. Goodrich Company Chemical Division
(42) Schülke & Mayr GmbH

Hair gel containing polymer as in Ex. B1 to B8 and Luviskol K90

| % | Raw material | Supplier | INCl |
|---|---|---|---|
| 0.50 | Carbopol ETD 2001 | (6) | Carbomer |
| 87.60 | Water, demin. | | Aqua dem. |
| 0.70 | Triethanolamin Care | (1) | Triethanolamin |
| 6.00 | Polymer B1 | (1) | |
| 5.00 | Luviskol K90 | (1) | PVP |
| q.s. | Perfume oil | | |
| q.s. | Cremophor CO 40 | (1) | PEG-40 Hydrogenated |
| | | | Castor Oil |
| 0.10 | Nipagin M | (34) | Methylparaben |
| 0.10 | Isopropyl myristate | (27) | Isopropyl Myristate |

Suppliers
(1) BASF Aktiengesellschaft
(6) B. F. Goodrich Company Chemical Division
(34) Nipa Laboratories Ltd.
(27) Cognis Deutschland GmbH

Hair gel containing polymer as in Ex. B1 to B8 and Luviquat Hold

| % | Raw material | Supplier | INCl |
|---|---|---|---|
| 10.00 | Polymer B1 to B8 | (1) | |
| 2.50 | Luviquat Hold. | (1) | Polyquaternium-46 |
| 15.00 | Ethanol 96% | | Alcohol |
| 70.30 | Water, demin. | | Aqua dem |
| 5.00 | Luviskol K90 | (1) | PVP |
| 0.10 | Perfume oil | | |
| 0.10 | Glycerol | (20) | Glycerin |
| 2.00 | Natrosol 250 HR | (4) | Hydroxyethylcellulose |

Suppliers
(1) BASF Aktiengesellschaft
(6) B. F. Goodrich Company Chemical Division
(20) Merck KGaA
(4) Aqualon GmbH

Hair gel containing polymer as in Ex. B1 to B8 and Amaze

| % | Raw material | Supplier | INCl |
|---|---|---|---|
| 6.00 | Polymer B1 | (1) | |
| 2.00 | Amaze | (72) | Corn Starch Modified |
| 0.50 | Hydagen HCMF | (27) | Chitosan |
| q.s. | Perfume oil | | |
| q.s. | Cremophor CO 40 | (1) | PEG-40 Hydrogenated |
| | | | Castor Oil |
| 0.10 | Abil 8843 | (44) | PEG-14 Dimethicone |
| 0.10 | Euxyl K100 | (42) | Benzyl Alcohol, Methyl- |
| | | | chloroisothiazolinone, |
| | | | Methylisothiazolinone |
| 91.40 | Water, demin. | | Aqua dem. |

Suppliers
(1) BASF Aktiengesellschaft
(6) B. F. Goodrich Company Chemical Division
(27) Cognis Deutschland GmbH
(42) Schülke & Mayr GmbH
(44) Th. Goldschmidt AG
(72) National Starch & Chemical Limited

Hair gel containing polymer as in Ex. B1 to B8 and Styleze CC-10

| % | Raw material | Supplier | INCl |
|---|---|---|---|
| 8.00 | Polymer B1 | (1) | |
| 5.00 | Styleze CC-10 | (65) | VP/DMAPA Acrylates |
| | | | Copolymer |
| 0.05 | AMP | (56) | Aminomethyl Propanol |
| 84.85 | Water, demin. | | Aqua dem |
| q.s. | Perfume oil | | |
| q.s. | Cremophor RH 40 | (1) | PEG-40 Hydrogenated |
| | | | Castor Oil |
| 0.10 | Dow Corning 190 | (16) | Dimethicone Copolyol |
| 0.10 | Euxyl K100 | (42) | |
| 2.00 | Klucel | (4) | Hydroxypropylcellulose |

Suppliers
(1) BASF Aktiengesellschaft
(4) Aqualon GmbH
(16) Dow Corning Corporation
(42) Schülke & Mayr GmbH
(56) Angus Chemical Company
(65) ISP Global Technologies Deutschland GmbH

Hair gel containing polymer as in Ex. 1g to 1l and Styleze 2000

| % | Raw material | Supplier | INCI |
|---|---|---|---|
| 6.00 | Polymer B1 | (1) | |
| 1.00 | Styleze 2000 | (65) | VP/Acrylates/Lauryl |
| | | | Methacrylate Copolymer |
| 0.26 | AMP | (56) | Aminomethyl Propanol |
| 90.64 | Water, demin. | | Aqua dem |
| q.s. | Perfume oil | | |
| q.s. | Cremophor RH 40 | (1) | PEG-40 Hydrogenated |
| | | | Castor Oil |
| 0.10 | Karion F Liquid | (20) | Sorbitol |
| 0.10 | Euxyl K100 | (42) | Benzyl Alcohol, Methyl- |
| | | | chloroisothiazolinone, |
| | | | Methylisothiazolinone |
| 2.00 | Hydroxypropyl guar | — | Hydroxypropylguar |

Suppliers
(1) BASF Aktiengesellschaft
(20) Merck KGaA
(42) Schülke & Mayr GmbH
(56) Angus Chemical Company
(65) ISP Global Technologies Deutschland GmbH

Hair gel containing polymer as in Ex. B1 to B8 and Allianz LT-120

| % | Raw material | Supplier | INCI |
|---|---|---|---|
| 0.50 | Ultrez 10 | (6) | Carbomer |
| 90.01 | Water, demin. | | Aqua dem. |
| 0.70 | Triethanolamine Care | (1) | Triethanolamine |
| 6.00 | Polymer B1 | (1) | |
| 2.00 | Allianz LT-120 | (61) | Acrylates/C1-2 |
| | | | Succinates/Hydroxy- |
| | | | acrylates Copolymer |
| 0.19 | AMP | (56) | Aminomethyl Propanol |
| q.s. | Perfume oil | | |
| q.s. | Cremophor CO 40 | (1) | PEG-40 Hydrogenated |
| | | | Castor Oil |
| 0.10 | Pluracare E400 | (1) | PEG-8 |
| 0.10 | Euxyl K100 | (42) | |
| 0.50 | Natrosol 250 HR | (4) | Hydroxyethylcellulose |

Suppliers
(1) BASF Aktiengesellschaft
(4) Aqualon GmbH
(6) B. F. Goodrich Company Chemical Division
(42) Schülke & Mayr GmbH
(56) Angus Chemical Company
(61) Röhm & Haas GmbH

Hair gel containing polymer as in Ex. B1 to B8 and Fixomer A30

| % | Raw material | Supplier | INCI |
|---|---|---|---|
| 7.00 | Polymer B1 | (1) | |
| 7.00 | Fixomer A30 | — | |
| 0.70 | Triethanolamine Care | (1) | Triethanolamine |
| q.s. | Perfume oil | | |
| q.s. | Cremophor CO 40 | (1) | PEG-40 Hydrogenated |
| | | | Castor Oil |
| 0.10 | D-Panthenol USP | (1) | Panthenol |
| 0.10 | Euxyl K100 | (42) | Benzyl Alcohol, Methyl- |
| | | | chloroisothiazolinone, |
| | | | Methylisothiazolinone |
| 84.90 | Water, demin. | | Aqua dem. |
| 1.00 | Sepigel 305 | (175) | |

Suppliers
(1) BASF Aktiengesellschaft
(42) Schülke & Mayr GmbH
(175) Seppic

Hair gel containing polymer as in Ex. B1 to B8 and PVF

| % | Raw material | Supplier | INCI |
|---|---|---|---|
| 0.50 | Carbopol 940 | (6) | Carbomer |
| 90.50 | Water, demin. | | Aqua dem. |
| 0.70 | Triethanolamine Care | (1) | Triethanolamine |
| 7.00 | Polymer B1 | (1) | |
| 1.00 | PVF | (72) | Polyvinylformamide |
| q.s. | Perfume oil | | |
| q.s. | Cremophor RH 40 | (1) | PEG-40 Hydrogenated |
| | | | Castor Oil |
| 0.10 | Euxyl K100 | (42) | Benzyl Alcohol, Methyl- |
| | | | chloroisothiazolinone, |
| | | | Methylisothiazolinone |
| 0.10 | Uvinul MC 80 | (1) | Ethylhexyl |
| | | | Methoxycinnamate |
| 0.10 | Abil 8843 | (44) | PEG-14 Dimethicone |

Suppliers
(1) BASF Aktiengesellschaft
(6) B. F. Goodrich Company Chemical Division
(42) Schülke & Mayr GmbH
(44) Th. Goldschmidt AG
(72) National Starch & Chemical Limited

Hair gel containing polymer as in Ex. B1 to B8

| % | Raw material | Supplier | INCI |
|---|---|---|---|
| 0.50 | Carbopol 940 | (6) | Carbomer |
| 88.50 | Water, demin. | | Aqua dem. |
| 0.70 | Triethanolamine Care | (1) | Triethanolamine |
| | | | Ethylenediamine |
| 10.00 | Polymer B1 | (1) | |
| q.s. | Perfume oil | | |
| q.s. | Cremophor CO 40 | (1) | PEG-40 Hydrogenated |
| | | | Castor Oil |
| 0.10 | Euxyl K100 | (42) | Benzyl Alcohol, Methyl- |
| | | | chloroisothiazolinone, |
| | | | Methylisothiazolinone |
| 0.10 | 1,2-Propylene glycol Care | (1) | Propylene Glycol |
| 0.10 | Isopropyl myristate | (27) | Isopropyl Myristate |

Suppliers
(1) BASF Aktiengesellschaft
(6) B. F. Goodrich Company Chemical Division
(27) Cognis Deutschland GmbH
(42) Schülke & Mayr GmbH

Hair gel containing polymer as in Ex. B1 to B8

| % | Raw material | Supplier | INCI |
|---|---|---|---|
| 10.00 | Polymer B1 | (1) | |
| 15.00 | Ethanol 96% | | |
| 72.70 | Water, demin. | | Aqua dem |
| 0.10 | Perfume oil | | |
| 0.10 | Glycerol | (20) | Glycerin |
| 0.10 | D-Panthenol USP | (1) | Panthenol |
| 2.00 | Natrosol 250 HR | (4) | Hydroxyethylcellulose |

Suppliers
(1) BASF Aktiengesellschaft
(6) B. F. Goodrich Company Chemical Division
(20) Merck KGaA
(4) Aqualon GmbH

Hair gel containing polymer as in Ex. B1 to B8

| % | Raw material | Supplier | INCI |
|---|---|---|---|
| 0.50 | Carbopol ETD 2001 | (6) | Carbomer |
| 88.50 | Water, demin. | | Aqua dem. |
| 0.70 | Triethanolamine Care | (1) | Triethanolamine |
| 10.00 | Polymer B1 | (1) | |
| q.s. | Perfume oil | | |
| q.s. | Cremophor CO 40 | (1) | PEG-40 Hydrogenated |
| | | | Castor Oil |
| 0.10 | Nipagin M | (34) | Methylparaben |
| 0.10 | Uvinul MC 80 | (1) | Ethylhexyl Methoxy- |
| | | | cinnamate |
| 0.10 | Abil 8843 | (44) | PEG-14 Dimethicone |

Suppliers
(1) BASF Aktiengesellschaft
(6) B. F. Goodrich Company Chemical Division
(34) Nipa Laboratories Ltd.
(44) Th. Goldschmidt AG

Hair gel containing polymer as in Ex. B1 to B8

| % | Raw material | Supplier | INCI |
|---|---|---|---|
| 0.50 | Ultrez 21 | (6) | Carbomer |
| 88.50 | Water, demin. | | Aqua dem. |
| 0.70 | Triethanolamine Care | (1) | Triethanolamine |
| 10.00 | Polymer B1 | (1) | |
| q.s. | Perfume oil | | |
| q.s. | Cremophor CO 40 | (1) | PEG-40 Hydrogenated |
| | | | Castor Oil |
| 0.10 | Nipagin M | (34) | Methylparaben |
| 0.10 | Uvinul P25 | (1) | PEG-25 PABA |
| 0.10 | Abil 8843 | (44) | PEG-14 Dimethicone |

Suppliers
(1) BASF Aktiengesellschaft
(6) B. F. Goodrich Company Chemical Division
(34) Nipa Laboratories Ltd.
(44) Th. Goldschmidt AG

Hair gel containing polymer as in Ex. B1 to B8

| % | Raw material | Supplier | INCI |
|---|---|---|---|
| 10.00 | Polymer B1 | (1) | |
| q.s. | Perfume oil | | |
| q.s. | Cremophor CO 40 | (1) | PEG-40 Hydrogenated |
| | | | Castor Oil |
| 0.10 | Palatinol A | (1) | Diethyl Phthalate |
| 0.10 | Luvitol EHO | (1) | Cetearyl Ethylhexanoate |
| 0.10 | Cetiol HE | (27) | PEG-7 Glyceryl Cocoate |
| 0.10 | Euxyl K100 | (42) | Benzyl Alcohol, Methyl- |
| | | | chloroisothiazolinone, |
| | | | Methylisothiazolinone |
| 87.70 | Water, demin. | | Aqua dem. |
| 2.00 | Luvigel EM | (1) | Caprylic/Capric Tri- |
| | | | glyceryde, Acrylates |
| | | | Copolymer |

Suppliers
(1) BASF Aktiengesellschaft
(27) Cognis Deutschland GmbH
(42) Schülke & Mayr GmbH

Setting solution containing polymer as in Ex. B1 to B8

| % | Raw material | Supplier | INCI |
|---|---|---|---|
| 62.60 | Ethanol 96%. | | Alcohol |
| 30.00 | Water, demin. | | Aqua dem. |
| 0.10 | Dow Corning 190 polyether | (16) | Dimethicone Copolyol |
| 0.10 | Perfume oil | | |
| 0.10 | Uvinul MC 80 | (1) | Ethylhexyl, Methoxy- |
| | | | cinnamate |
| 0.10 | D-Panthenol USP | (1) | Panthenol |
| 7.00 | Polymer B1 | (1) | |

Suppliers
(1) BASF Aktiengesellschaft
(16) Dow Corning Corporation

Setting solution containing polymer as in Ex. B1 to B8

| % | Raw material | Supplier | INCI |
|---|---|---|---|
| 0.10 | Dow Corning 190 polyether | (16) | Dimethicone Copolyol |
| 0.05 | Dow Corning 344 fluid | (16) | Cyclomethicone |
| q.s. | Perfume oil | | |
| 53.85 | Ethanol 96% | | Alcohol |
| 40.00 | Water, demin. | | Aqua dem. |
| 6.00 | Polymer B1 | (1) | |

Suppliers
(1) BASF Aktiengesellschaft
(16) Dow Corning Corporation

Setting solution containing polymer as in Ex. B1 to B8

| % | Raw material | Supplier | INCI |
|---|---|---|---|
| 0.10 | D-Panthenol USP | (1) | Panthenol |
| 0.10 | Nutrilan Keratin W | | |
| 0.10 | Elastin PG 2000 | | Hydrolyzed Elastin |
| 0.40 | Uvinul M 40 | (1) | Benzophenone-3 |
| 10.00 | Water, demin. | | Aqua dem. |
| 84.30 | Ethanol 96% | | Alcohol |
| q.s. | Perfume oil | | |
| 5.00 | Polymer B1 | (1) | |

Suppliers
(1) BASF Aktiengesellschaft

Setting solution containing polymer as in Ex. B1 to B8 and Luviquat FC 550

| % | Raw material | Supplier | INCI |
|---|---|---|---|
| 4.00 | Polymer B1 | (1) | Polyvinylcaprolactam |
| 3.50 | Luviquat FC 550 | (1) | Polyquaternium-16 |
| 72.20 | Ethanol 96% | | Alcohol |
| 20.00 | Water, demin. | | Aqua dem. |
| q.s. | Perfume oil | | |

Suppliers
(1) BASF Aktiengesellschaft

Setting solution containing polymer as in Ex. B1 to B8

| % | Raw material | Supplier | INCI |
|---|---|---|---|
| 4.00 | Polymer B1 | (1) | |
| 0.20 | Pluracare E 400 | (1) | PEG-8 |
| 0.10 | Perfume oil | | |
| 10.00 | Water, demin. | | |
| 85.70 | Ethanol 96% | | Alcohol |

Suppliers
(1) BASF Aktiengesellschaft

Pump spray containing polymer as in Ex. B1 to B8

| % | Raw material | Supplier | INCI |
|---|---|---|---|
| 26.00 | Polymer B1 | (1) | |
| 73.70 | Ethanol 96% | | Alcohol |
| 0.10 | Perfume oil | | |
| 0.10 | Uvinul MC 80 | (1) | Ethylhexyl Methoxy- |
| | | | cinnamate |
| 0.10 | Dow Corning 190 | (16) | PEG/PPG-18/18 Dimethi- |
| | | | cone |

Suppliers
(1) BASF Aktiengesellschaft
(16) Dow Corning Corporation

Pump spray containing polymer as in Ex. B1 to B8

| % | Raw material | Supplier | INCI |
|---|---|---|---|
| 26.00 | Polymer B1 | (1) | |
| 4.00 | Luviskol Plus | (1) | Polyvinylcaprolactam |
| 69.60 | Ethanol 96% | | Alcohol |
| 0.10 | Uvinul MC 80 | (1) | Ethylhexyl Methoxy- |
| | | | cinnamate |
| 0.10 | Dow Corning 344 | (16) | Cyclomethicone |
| 0.10 | Dow Corning 556 | (16) | Phenyl Trimethicone |

Suppliers
(1) BASF Aktiengesellschaft
(16) Dow Corning Corporation

Aerosol spray NON VOC containing polymer as in Ex. B1 to B8

| % | Raw material | Supplier | INCI |
|---|---|---|---|
| 13.00 | Polymer B1 | (1) | |
| 0.10 | Perfume oil | | |
| 0.10 | 1,2-Propylene glycol Care | (1) | Propylene Glycol |
| 0.10 | Citroflex 2 | (53) | Triethyl Citrate |
| 46.70 | Water, demin. | | Aqua dem |
| 40.00 | HFC 152A | — | Hydrofluorocarbon 152a |

Suppliers
(1) BASF Aktiengesellschaft.
(53) Pfizer Chemie

Aerosol spray NON VOC containing polymer as in Ex. B1 to B8 and Luviset CAN

| % | Raw material | Supplier | INCI |
|---|---|---|---|
| 10.00 | Polymer B1 | (1) | |
| 2.00 | Luviset CAN | (1) | VA/Crotonates/Vinyl |
| | | | Neodecanoate Copolymer |
| 0.16 | AMP | (56) | Aminomethyl Propanol |
| 0.10 | Perfume oil | | |
| 0.10 | Phytantriol | (1) | Phytantriol |
| 52.64 | Water, demin. | | Aqua dem. |
| 35.00 | HFC 152A | — | Hydrofluorocarbon 152a |

Suppliers
(1) BASF Aktiengesellschaft
(56) Angus Chemical Company

Aerosol spray VOC 55 containing polymer as in Ex. B1 to B8 and Luviset P.U.R.

| % | Raw material | Supplier | INCI |
|---|---|---|---|
| 7.00 | Polymer B1 | (1) | |
| 7.00 | Luviset P.U.R. | (1) | Polyurethane-1 |
| | | | Neodecanoate Copolymer |
| 14.30 | Ethanol absolute | | Alcohol |
| 36.50 | Water, demin. | | Aqua dem. |
| 0.10 | 1,2-Propylene glycol Care | (1) | Propylene Glycol |
| 0.10 | Perfume oil | | |
| 40.00 | DME | — | Dimethyl ether |

Suppliers
(1) BASF Aktiengesellschaft

Aerosol spray VOC 55 containing polymer as in Ex. B1 to B8 and Luviskol Plus

| % | Raw material | Supplier | INCI |
|---|---|---|---|
| 10.00 | Polymer B1 | (1) | |
| 5.00 | Luviskol Plus | (1) | Polyvinylcaprolactam |
| 17.00 | Ethanol absolute | | Alcohol |
| 32.80 | Water, demin. | | Aqua dem. |
| 0.10 | Niacinamide | — | Niacinamide |
| 0.10 | Perfume oil | | |
| 35.00 | DME | — | Dimethyl ether |

Suppliers
(1) BASF Aktiengesellschaft

Aerosol spray VOC 80 containing polymer as in Ex. B1 to B8 and Luvimer 100P

| % | Raw material | Supplier | INCI |
|---|---|---|---|
| 10.00 | Polymer B1 | (1) | |
| 1.00 | Luvimer 100P | (1) | Acrylates Copolymer |
| 0.24 | AMP | (56) | Aminomethyl Propanol |
| 35.00 | Ethanol absolute | | Alcohol |
| 8.56 | Water, demin. | | Aqua dem. |
| 0.10 | Belsil CM040 | (156) | Cyclopentasiloxane |
| 0.10 | Perfume oil | | |
| 10.00 | n-Butane | — | Butane |
| 35.00 | DME | — | Dimethyl ether |

Suppliers
(1) BASF Aktiengesellschaft
(56) Angus Chemical Company
(156) Wacker Chemie GmbH

Aerosol spray VOC 80 containing polymer as in Ex. B1 to B8 and Luviskol VA37

| % | Raw material | Supplier | INCI |
|---|---|---|---|
| 10.00 | Polymer B1 | (1) | |
| 4.00 | Luviskol VA37 | (1) | VP/VA Copolymer |
| 38.00 | Ethanol absolute | | Alcohol |
| 7.70 | Water, demin. | | Aqua dem. |
| 0.10 | D-Panthenol USP | (1) | Panthenol |
| 0.10 | Dow Corning 556 | (16) | Phenyl Trimethicone |
| 0.10 | Perfume oil | | |
| 40.00 | DME | — | Dimethyl ether |

Suppliers
(1) BASF Aktiengesellschaft
(16) Dow Corning Corporation

Aerosol spray without the addition of water containing polymer as in Ex. B1 to B8 and Luviflex Silk

| % | Raw material | Supplier | INCI |
|---|---|---|---|
| 7.00 | Polymer B1 | (1) | |
| 4.00 | Luviflex Silk. | (1) | PEG/PPG-25/25 Dimethi- |
| | | | cone/Acrylates Copolymer |
| 0.47 | AMP | (56) | Aminomethyl Propanol |
| 48.23 | Ethanol absolute | | Alcohol |
| 0.10 | Palatinol A | (1) | Diethyl Phthalate |
| 0.10 | D-Panthenol USP | (1) | Panthenol |
| 0.10 | Perfume oil | | |
| 10.00 | Propane/Butane | — | Propane/Butane |
| 30.00 | DME | — | Dimethyl ether |

Suppliers
(1) BASF Aktiengesellschaft
(56) Angus Chemical Company

Aerosol spray without the addition of water containing polymer as in Ex. B1 to B8 and Amphomer

| % | Raw material | Supplier | INCI |
|---|---|---|---|
| 10.00 | Polymer B1 | (1) | |
| 1.00 | Amphomer 28-4910 | (72) | Acrylates Copolymer |
| 0.17 | AMP | (56) | Aminomethyl Propanol |
| 43.53 | Ethanol absolute | | Alcohol |
| 0.10 | Dow Corning 193 | (16) | PEG-12 dimethicone |
| 0.10 | Dow Corning 556 | (16) | Phenyl Trimethicone |
| 0.10 | Perfume oil | | |
| 45.00 | DME | — | Dimethyl ether |

Suppliers
(1) BASF Aktiengesellschaft
(16) Dow Corning Corporation
(56) Angus Chemical Company
(72) National Starch & Chemical Limited

Mixing procedures

Pump Mousse Hair Setting Composition

| | | |
|---|---|---|
| 3.00 | Polymer B1 | |
| 1.00 | Luviquat Mono CP | Hydroxyethyl Cetyldimonium |
| | | Phosphate |
| 0.20 | Cremophor A 25 | Ceteareth-25 |
| 0.40 | Perfume oil PC | |
| | 910.781/Cremophor | |
| 95.40 | Water, demin. | Aqua dem. |
| q.s. | Preservative | |

Preparation:

Prepare a uniform mixture from the components and bottle in a pump mousse bottle.

PUMP SPRAY

| A | q.s. | Cremophor CO 40 | PEG-40 Hydrogenated |
|---|---|---|---|
| | | | Castor Oil |
| | q.s. | Perfume oil | |
| | 75.50 | Water, demin. | Aqua dem. |
| | 7.30 | Polymer B1 | |
| B | 1.00 | 1,2-Propylene glycol Care | Propylene Glycol |
| | 0.20 | Uvinul P 25 | PEG-25 PABA |
| | 1.00 | Luviquat HM 552 | Polyquaternium-16 |
| | 15.00 | Ethanol 96% | Alcohol |

Preparation:

Stir phase A. Add the components of Phase B one after the other and distribute uniformly. Then bottle everything.

STYLING WATER

| | | | |
|---|---|---|---|
| A | 0.70 | Cremophor CO 40 | PEG-40 Hydrogenated |
| | | | Castor Oil |
| | 0.20 | Perfume oil | |
| | 75.10 | Water, demin. | Aqua dem. |
| | 7.30 | Polymer B1 | |
| B | 1.00 | 1,2-Propylene glycol Care | Propylene Glycol |
| | 0.50 | Luviquat Care | Polyquaternium-44 |
| | 0.20 | Uvinul P 25 | PEG-25 PABA |
| | 15.00 | Ethanol 96% | Alcohol |

Preparation:

Stir phase A. Add the components of phase B one after the other and distribute uniformly. Then bottle everything.

HAIR MOUSSE

| | | | |
|---|---|---|---|
| A | 0.70 | Cremophor CO 40 | PEG-40 Hydrogenated |
| | | | Castor Oil |
| | 0.20 | Perfume oil | |
| | 78.50 | Water, demin. | Aqua dem. |
| B | 0.50 | Luviquat Mono LS | Cocotrimonium Methosulfate |
| | 6.70 | Polymer B1 | |
| | 2.50 | Luviquat Hold | Polyquaternium-46 |
| | 0.20 | Uvinul P 25 | PEG-25 PABA |
| | 0.50 | Pluracare E 400 | PEG-8 |
| | 0.20 | Cremophor A 25 | Ceteareth-25 |
| | q.s. | Preservative | |
| C | 10.00 | Propane/butane 3.5 | Propane/Butane |
| | | bar (20° C.) | |

Preparation:

Stir phase A. Add the components of phase B one after the other and distribute uniformly. Bottle with phase C.

STYLING MOUSSE

| | | | |
|---|---|---|---|
| A | 2.00 | Luviquat Mono LS | Cocotrimonium methosulfate |
| | q.s. | Perfume oil | |
| B | 62.85 | Water, demin. | Aqua dem. |
| | 7.00 | Polymer B1 | |
| | 2.00 | Luviquat PQ 11 | Polyquaternium-11 |
| | 0.20 | Cremophor A 25 | Ceteareth-25 |
| | 0.50 | D-Panthenol USP | Panthenol |
| | 0.05 | Uvinul MS 40 | Benzophenone-4 |
| | 0.20 | Dow Corning 949 Cationic | |
| | 15.00 | Ethanol 96% | Alcohol |
| C | 0.20 | Natrosol 250 HR | Hydroxyethylcellulose |
| D | 10.00 | Propane/butane 3.5 bar | Propane/Butane |
| | | (20° C.) | |

Preparation:

Mix phase A. Add the components of phase B one after the other and mix. Add phase C and stir until distributed uniformly. Adjust the pH to 6-7. Bottle with phase D.

SETTING FOAM

| | | | |
|---|---|---|---|
| A | 2.00 | Luviquat Mono LS | Cocotrimonium methosulfate |
| | q.s. | Perfume oil | |
| B | 83.13 | Water, demin. | Aqua dem. |
| | 0.47 | AMP | Aminomethyl Propanol |
| | 0.20 | Preservative | |
| | 0.20 | Abil B 8843 | Dimethicone Copolyol |
| C | 4.00 | Polymer B1 | |
| D | 10.00 | Propane/butane 3.5 bar | Propane/Butane |
| | | (20° C.) | |

Preparation:

Mix phase A. Weigh in phase B and dissolve until clear. Stir phase B into phase A. Add phase C and stir. Bottle with phase D.

WETLOOK SETTING FOAM

| | | | |
|---|---|---|---|
| A | 3.00 | Luviquat Mono LS | Cocotrimonium methosulfate |
| | 0.20 | Perfume oil | |
| B | 78.80 | Water, demin. | Aqua dem. |
| C | 5.00 | Glycerol 87% | Glycerin |
| | q.s. | Preservative | |
| | 3.00 | Polymer B1 | |
| D | 10.00 | Propane/butane 3.5 bar | Propane/Butane |
| | | (20° C.) | |

Preparation:

Mix phase A. Stir phase B into phase A. Add phase C. Bottle with phase D.

FOAM CONDITIONER

| | | |
|---|---|---|
| 5.00 | Luviquat PQ 11 | Polyquaternium-11 |
| 5.00 | Polymer B1 | |
| 0.50 | Luviquat Mono CP | Hydroxyethyl Cetyldimonium Phosphate |
| 10.00 | Ethanol abs. | Alcohol |
| 0.40 | “Carina” perfume | |
| | oil/Cremophor RH | |
| q.s. | Preservative | |
| 69.10 | Water, demin. | Aqua dem. |
| 10.00 | Propane/butane | Propane/Butane |

Preparation:

Weigh everything together, stir until homogeneously distributed, bottle.

SHEEN HAIR WAX

| | | |
|---|---|---|
| 5.00 | Luvitol EHO | Cetearyl Octanoate |
| 5.00 | Castor oil | Castor (Ricinus |
| | | Communis) Oil |
| 17.00 | Vaseline | Petrolatum |
| 7.00 | TeCero wax 1030 K | Microcrystalline Wax |
| 6.00 | Beeswax 3044 PH | Bees Wax |
| 5.00 | Polymer B1 (anhydrous) | |
| 3.00 | Uvinul MBC 95 | 4-Methylbenzylidene |
| | | Camphor |
| 2.00 | Uvinul BMBM | Butyl Methoxydibenzoyl- |
| | | methane |
| 0.10 | Phytantriol | Phytantriol |
| 0.50 | Phenoxyethanol | Phenoxyethanol |
| 48.40 | Paraffin oil, high viscosity | Mineral Oil |
| .00 | Dow Corning 556 fluid | Phenyl Trimethicone |
| q.s. | Perfume oil | |

Preparation:

Weigh in the components of phase A and melt.

COLOR BALSAM

| | | | |
|---|---|---|---|
| A | 1.50 | Cremophor A 6 | Ceteareth-6, Stearyl Alcohol |
| | 1.50 | Cremophor A 25 | Ceteareth-25 |
| | 3.00 | Cetylstearyl alcohol | Cetearyl Alcohol |
| | 6.00 | Luvitol EHO | Cetearyl Octanoate |
| | 0.30 | Phytantriol | Phytantriol |
| B | 7.70 | Luviquat Care | Polyquaternium-44 |
| | 6.00 | Polymer B1 | |
| | 2.00 | 1,2-Propylene glycol Care | Propylene Glycol |
| | 1.00 | D-Panthenol USP | Panthenol |
| | q.s. | Preservative | |
| | 70.87 | Water, demin. | Aqua dem. |
| C | 0.05 | Basic Violet 14 | C.I. 42510, Basic Violet 14 |
| | 0.08 | Basic Red 76 | C.I. 12245, Basic Red 76 |
| | q.s. | Perfume oil | |
| | q.s. | Citric acid | Citric Acid |

Preparation:

Heat phases A and B separately to about 80° C. Stir phase B into phase A with homogenization, briefly after-homogenize. Cool to about 40° C., add phase C and briefly homogenize again. Adjust the pH to 6 to 7.

HAIR REPAIR TREATMENT

| | | | |
|---|---|---|---|
| A | 0.20 | Luvitol EHO | Cetearyl Octanoate |
| | 3.00 | Polymer B1 | |
| | 0.10 | Phytantriol | Phytantriol |
| | 2.00 | Cremophor CO 40 | PEG-40 Hydrogenated Castor Oil |
| B | q.s. | Perfume oil | |
| | 2.00 | Luviquat Mono LS | Cocotrimonium Methosulfate |
| C | 79.70 | Water, demin. | Aqua dem. |
| D | 2.00 | Luviquat FC 905 | Polyquaternium-16 |
| | 1.00 | Silicone oil SF 1288 | Dimethicone Copolyol |
| | q.s. | Preservative | |
| | 10.00 | Ethanol 96% | Alcohol |
| | q.s. | Citric acid | Citric Acid |

Preparation:

Separately mix the phases A and B. Stir phase C into phase B. Stir the solution of phases B and C into phase A. Add phase D and stir until thickened. Adjust the pH to 4 to 5.

HAIR GUM

| | | | |
|---|---|---|---|
| A | 0.50 | Glucamate SSE-20 | PEG-20 Methyl Glucose |
| | | | Sesquistearate |
| | q.s. | Cremophor CO 40 | PEG-40 Hydrogenated |
| | | | Castor Oil |
| | q.s. | Perfume oil | |
| | 30.00 | Water, demin. | Aqua dem. |
| B | 10.00 | Luviquat Hold | Polyquaternium-46 |
| | 2.00 | Luviskol K 90 | PVP |
| | 6.00 | Polymer B1 | |
| | 0.30 | Germall 115 | Imidazolidinyl Urea |
| | 0.10 | Euxyl K 100 | Benzyl Alcohol, Methyl- |
| | | | chloroisothiazolinone, |
| | | | Methylisothiazone |
| | 0.50 | D-Panthenol USP | Panthenol |
| | 5.00 | Pluracare E 6000 | PEG 90 |
| | 3.00 | 1,2-Propylene glycol Care | Propylene Glycol |
| | 40.10 | Water, demin. | Aqua dem. |
| C | 2.50 | Natrosol 250 HR | Hydroxyethylcellulose |

Preparation:

Solubilize phase A. Dissolve phase B and stir into phase A. Stir phase C into the solution of phases A and B.

SILKY HAIR COCKTAIL

| | | | |
|---|---|---|---|
| A | 3.00 | Luvigel EM | Caprylic/Capric |
| | | | Triglyceride, |
| | | | Acrylates Copolymer |
| | 3.00 | Polymer B1 (anhydrous) | |
| | 0.50 | Wacker Belsil DMC 6031 | Dimethicone Copolyol |
| | 2.00 | Wacker Belsil DM 1000 | Dimethicone |
| | 3.00 | Wacker Belsil CM 1000 | Cyclomethicone, |
| | | | Dimethiconol |
| | 2.00 | Wacker Belsil ADM 6057E | Amodimethicone, |
| | | | Cetrimonium Chloride, |
| | | | Trideceth-10 |
| | 2.00 | Wacker Belsil PDM 200 | Phenyl Trimethicone |
| | 1.00 | Macadamia nut oil | Macadamia (Ternifolia) |
| | | | Nut Oil |
| | 0.50 | Vitamin E acetate | Tocopheryl Acetate |
| | 1.00 | Cremophor CO 40 | PEG-40 Hydrogenated |
| | | | Castor Oil |
| | q.s. | Perfume oil | |
| B | 77.54 | Water, demin. | Aqua dem. |
| | 0.46 | AMP | Aminomethyl Propanol |
| | 4.00 | Luviflex Silk | PEG/PPG-25/25 |
| | | | Dimethicone/ |
| | | | Acrylates Copolymer |
| | q.s. | Preservative | |

Preparation:

Mix the components of phase A. Dissolve phase B. Stir phase B into phase A with homogenization.

OIL SHEEN MOISTURIZER

| | | | |
|---|---|---|---|
| A | 2.00 | Cetyl alcohol | Cetyl Alcohol |
| | 1.00 | Solan ELD | PEG-75 Lanolin |
| | 4.00 | Glycerol monostearate | Glyceryl Stearate |
| | 1.00 | Cremophor A 25 | Ceteareth-25 |
| | 4.00 | Luvitol EHO | Cetearyl Octanoate |
| B | 10.00 | Glycerol 87% | Glycerin |
| | 5.00 | Polymer B1 | |
| | 2.00 | 1,2-Propylene glycol Care | Propylene Glycol |
| | 1.00 | Luviquat Mono LS | Cocotrimonium Methosulfate |
| | 1.50 | Silicone microemulsion | Trimethylsilylamodimethicone, |
| | | | SM 2115 Octoxynol-40, |
| | | | Isolaureth-6, Glycerin |
| | 1.00 | Cremophor PS 20 | Polysorbate 20 |
| | 67.00 | Water, demin. | Aqua dem. |
| C | 0.50 | D-Panthenol USP | Panthenol |
| | q.s. | Preservative | |
| | q.s. | Perfume oil | |
| | q.s. | Citric acid | Citric Acid |

Preparation:

Heat phases A and B separately to about 80° C. Stir phase B into phase A and homogenize. Cool to about 40° C., add phase C and homogenize well again.

SETTING CREAM HIGH GLOSS

| | | | |
|---|---|---|---|
| A | 5.00 | Cetyl alcohol | Cetyl Alcohol |
| | 10.00 | Tegin | Glyceryl Stearate SE |
| | 5.00 | Isopropyl myristate | Isopropyl Myristate |
| | q.s. | Preservative | |
| | 1.00 | Dow Corning 200 fluid | Dimethicone |
| B | 5.00 | Glycerol 87% | Glycerin |
| | 5.00 | Polymer B1 | |
| | 0.20 | Edeta BD | Disodium EDTA |
| | 2.00 | Luviskol K 30 | PVP |
| | 66.80 | Water, demin. | Aqua dem. |
| C | q.s. | Perfume oil | |

Preparation:

Heat phases A and B separately to about 80° C. Stir phase B into phase A and homogenize. Cool to about 40° C., add phase C and homogenize briefly again.

PERMANENT WAVE

| | | | |
|---|---|---|---|
| A | 70.95 | Water, demin. | Aqua dem. |
| | 3.00 | Polymer B1 | |
| | 0.20 | Tego Betaine L 7 | Cocamidopropyl Betaine |
| | 0.20 | Cremophor PS 20 | Polysorbate 20 |
| | 1.25 | Luviquat FC 905 | Polyquaternium-16 |
| | 0.20 | Edeta BD | Disodium EDTA |
| | 0.20 | Natrosol 250 HR | Hydroxyethylcellulose |
| B | 8.00 | Thioglycolic acid 80% | Thioglycolic Acid |
| C | 11.00 | Ammonia solution 25% | Ammonium Hydroxide |
| D | 5.00 | Ammonium carbonate | Ammonium Carbonate |

Preparation:

Weigh in the components of phase A and mix. Stir phase B into phase A.

NEUTRALIZER FOR PERMANENT WAVE

| | | | |
|---|---|---|---|
| A | 1.00 | Cremophor CO 40 | PEG-40 Hydrogenated Castor Oil |
| | 0.20 | Perfume oil | |
| | 2.00 | Polymer B1 | |
| | 91.60 | Water, demin. | Aqua dem. |
| B | 0.20 | Tego Betaine L 7 | Cocamidopropyl Betaine |
| | 0.20 | Cremophor A 25 | Ceteareth-25 |
| | 2.50 | Luviquat FC 905 | Polyquaternium-16 |
| | q.s. | Preservative | |
| C | 2.30 | Hydrogen peroxide 30% | Hydrogen Peroxide |
| D | q.s. | Phosphoric acid 85% | Phosphoric Acid |

Preparation:

Solubilize phase A. Add the components of phase B one after the other and mix. Add phase C and stir again. Adjust the pH to 3.0 to 3.5.

Water-resistant mascara
A

| | |
|---|---|
| 46.7 | Dist. Water |
| 3.0 | Lutrol E 400 (PEG-8) |
| 0.5 | Xanthan gum |
| q.s. | Preservative |
| 0.1 | Imidazolidinylurea |
| 1.3 | Tetrahydroxypropylethylenediamine |

B

| | |
|---|---|
| 8.0 | Carnauba wax |
| 4.0 | Beeswax |
| 4.0 | Isoeicosane |
| 4.0 | Polyisobutenes |
| 5.0 | Stearic acid |
| 1.0 | Glyceryl stearate |
| q.s. | Preservative |
| 2.0 | Benzyl laurate |

C

| | |
|---|---|
| 10.0 | Iron oxide/Al powder/silica (e.g. Sicopearl Fantastico |
| | Gold ™ from BASF) |

E

| | |
|---|---|
| 10.0 | Polymer B1 according to the invention |

Preparation:

Heat phase A and phase B separately to 85° C. Maintain the temperature and add phase C to phase A and homogenize until the pigments are uniformly distributed. Add phase B to phases AC and homogenize for 2-3 minutes. Then add phase E and stir slowly. Allow everything to cool to room temperature.

Styling Mousses

02/00483 STYLING MOUSSE WITH LUVISET CLEAR

| A | 2.00 | Luviquat Mono LS | Cocotrimonium Methosulfate |
|---|---|---|---|
| | q.s. | Perfume oil | |
| B | 78.00 | Water, demin. | Aqua dem. |
| | 2.50 | Luviset Clear | VP/Methacrylamide/Vinyl Imidazole |
| | | | Copolymer |
| | 7.50 | Polymer according to the invention from example B1 | |
| | q.s. | Preservative | |
| C | 10.00 | Propane/butane 3.5 bar | Propane/Butane |
| | | (20° C.) | |

02/00485 STYLING MOUSSE WITH LUVIQUAT STYLE

| A | 2.00 | Luviquat Mono LS | Cocotrimonium Methosulfate |
|---|---|---|---|
| | q.s. | Perfume oil | |
| B | 78.00 | Water, demin. | Aqua dem. |
| | 2.50 | Luviquat Style | Polyquaternium-16 |
| | 7.50 | Polymer according to the invention from example B1 | |
| | q.s. | Preservative | |
| C | 10.00 | Propane/butane 3.5 bar | Propane/Butane |
| | | (20° C.) | |

02/00491 STYLING MOUSSE WITH LUVISKOL VA 64

| A | 2.00 | Luviquat Mono LS | Cocotrimonium Methosulfate |
|---|---|---|---|
| | q.s. | Perfume oil | |
| B | 82.50 | Water, demin. | Aqua dem. |
| | 3.00 | Luviskol VA 64 P | PVP/VA Copolymer |
| | 2.50 | Polymer according to the invention from example B1 | |
| | q.s. | Preservative | |
| C | 10.00 | Propane/butane 3.5 bar | Propane/butane |
| | | (20° C.) | |

STYLING MOUSSE WITH POLYQUATERNIUM-4

| A | 2.00 | Luviquat Mono LS | Cocotrimonium Methosulfate |
|---|---|---|---|
| | q.s. | Perfume oil | |
| B | 78.00 | Water, demin. | Aqua dem. |
| | 5.00 | Celquat H100 or L200 | Polyquaternium-4 |
| | 5.00 | Polymer according to the invention from example B1 | |
| | q.s. | Preservative | |
| C | 10.00 | Propane/butane 3.5 bar | Propane/Butane |
| | | (20° C.) | |

STYLING MOUSSE WITH PVP/DMAPA/Acrylates Copolymer

| A | 2.00 | Luviquat Mono LS | Cocotrimonium Methosulfate |
|---|---|---|---|
| | q.s. | Perfume oil | |
| B | 78.00 | Water, demin. | Aqua dem. |
| | 5.00 | Styleze CC10 | PVP/DMAPA/Acrylates |
| | | | Copolymer |
| | 5.00 | Polymer according to the invention from example B1 | |
| | q.s. | Preservative | |
| C | 10.00 | Propane/butane 3.5 bar | Propane/butane |
| | | (20° C.) | |

STYLING MOUSSE WITH POLYQUATERNIUM-55

| A | 2.00 | Luviquat Mono LS | Cocotrimonium Methosulfate |
|---|---|---|---|
| | q.s. | Perfume oil | |
| B | 78.00 | Water, demin. | Aqua dem. |
| | 5.00 | Styleze W-20 | Polyquaternium-55 |
| | 5.00 | Polymer according to the invention from example B1 | |
| | q.s. | Preservative | |
| C | 10.00 | Propane/butane 3.5 bar | Propane/butane |
| | | (20° C.) | |

STYLING MOUSSE WITH POLYQUATERNIUM-10

| A | 2.00 | Luviquat Mono LS | Cocotrimonium Methosulfate |
|---|---|---|---|
| | q.s. | Perfume oil | |
| B | 78.00 | Water, demin. | Aqua dem. |
| | 5.00 | UCARE Polymer IR400 | Polyquaternium-10 |
| | 5.00 | Polymer according to the invention from example B1 | |
| | q.s. | Preservative | |
| C | 10.00 | Propane/butane 3.5 bar | Propane/butane |
| | | (20° C.) | |

STYLING MOUSSE WITH ACRYLATES COPOLYMER

| A | 2.00 | Luviquat Mono LS | Cocotrimonium Methosulfate |
|---|---|---|---|
| | q.s. | Perfume oil | |
| B | 78.00 | Water, demin. | Aqua dem. |
| | 2.50 | Luviflex Soft | Acrylates Copolymer |
| | 7.50 | Polymer according to the invention from example B1 | |
| | q.s. | Preservative | |
| C | 10.00 | Propane/butane 3.5 bar | Propane/butane |
| | | (20° C.) | |

STYLING MOUSSE WITH CORN STARCH MODIFIED

| A | 2.00 | Luviquat Mono LS | Cocotrimonium Methosulfate |
|---|---|---|---|
| | q.s. | Perfume oil | |
| B | 78.00 | Water, demin. | Aqua dem. |
| | 5.00 | Amaze 28-1890 | Corn Starch Modified |
| | 5.00 | Polymer according to the invention from example B1 | |
| | q.s. | Preservative | |
| C | 10.00 | Propane/butane 3.5 bar | Propane/butane |
| | | (20° C.) | |

STYLING MOUSSE WITH VP/Dimethylaminoethyl
Methacrylate Copolymer

| A | 2.00 | Luviquat Mono LS | Cocotrimonium Methosulfate |
|---|---|---|---|
| | q.s. | Perfume oil | |
| B | 78.00 | Water, demin. | Aqua dem. |
| | 5.00 | Styleze 845 | VP/Dimethylaminoethyl |
| | | | Methacrylate Copolymer |
| | 5.00 | Polymer according to the invention from example B1 | |
| | q.s. | Preservative | |
| C | 10.00 | Propane/butane 3.5 bar | Propane/butane |
| | | (20° C.) | |

The preparation of the cosmetic formulations with the polymers from example B1-B8 takes place analogously to the formulations described for polymer B1.

1-4. (canceled)

5. A cosmetic preparation comprising a polymer, water and/or an alcohol and at least one further additive customary in cosmetics wherein the polymer comprises:
30-65% by weight of N-vinylcaprolactam,
35-65% by weight of a monomer mixture of N-vinylimidazole and vinylpyrrolidone, where the weight ratio of N-vinylimidazole to vinylpyrrolidone is between 0.085 and 0.30,
0-15% by weight of a nitrogen-containing quaternizable monomer (A), which is different from N-vinylimidazole, where the sum of monomer (A) and N-vinylimidazole is less than 18% by weight.

6. The cosmetic preparation as claimed in claim 5, wherein the cosmetic preparation comprises 0.001 to 20% by weight of the polymer and 20 to 99.95% by weight of water and/or the alcohol.

7. The cosmetic preparation as claimed in claim 5, wherein the at least one further additive customary in cosmetics is at least one additive selected group consisting of a propellant, an antifoam, a surfactant, an emulsifier, a foam formed, a solubilizer, an anionic polymer, a cationic polymer, an amphoteric polymer, a neutral polymer, a thickener, a silicone compound, an oil, a wax, a fatty acid, an alcohol, a preservative, a perfume, an opacifier, an active ingredient, a UV filter, panthenol, collegen, a vitamin, a protein hydrolysate, an alpha-hydrocarboxylic acid, a beta-hydroxylic acid, a stabilizer, a pH regulator, a dye, a viscosity regulator, a gel former, a dye, a salt, a humectant, a refatting agent and a complexing agent.

8. A skin cosmetic preparation comprising the cosmetic preparation as claimed in claim 5, wherein the skin cosmetic preparation is a soap product, a nailcare product, a skin protection product, a skincare product, a personal hygiene product, a footcare product, a deodorant, a photoprotective product, a repellant, a shaving product, a hair-removal product, an antiacne product, a make-up product, a mascara product, a lipstick product, an eyeshadow product, a kohl pencil product, an eyeliner product, a blusher product, a powder product or an eyebrow pencil.

9. A hair cosmetic preparation comprising the cosmetic preparation as claimed in claim 5, wherein the hair cosmetic preparation is a hair mousse, a hair gel, a hair lotion, a hair rinse, a hair shampoo, a hair emulsion, a hair-end fluid, a neutralizer for permanent wave, a hair colorant, a hair bleach, a hot-oil treatment for hair, a hair conditioner, a hair setting lotion or a hair spray.

10. A hair or skin cosmetic preparation comprising the cosmetic preparation as claimed in claim 5 and a gelling agent.

11. A method of treating skin comprising applying the skin cosmetic preparation as claimed in claim 8 to skin.

12. A method of treating hair comprising applying the hair cosmetic preparation as claimed in claim 9 to hair.

13. A method of setting hair comprising applying the hair cosmetic preparation as claimed in claim 9 to hair.

14. A method of treating hair or skin comprising applying the hair or skin cosmetic preparation as claimed in claim 10 to hair or skin.

15. The skin cosmetic preparation as claimed in claim 8, wherein the skin cosmetic preparation is in the form of a water-in-oil emulsion or an oil-in-water emulsion.
